(19) United States
(12) Patent Application Publication
(10) Pub. No.: US 2024/0238532 A1
(43) Pub. Date: Jul. 18, 2024

(54) NEEDLE SHIELD REMOVER

(71) Applicant: SHL MEDICAL AG, Zug (CH)

(72) Inventor: Daniel CARLSSON, Nacka Strand (SE)

(21) Appl. No.: 18/558,002

(22) PCT Filed: Apr. 19, 2022
(86) PCT No.: PCT/EP2022/060244

§ 371 Date: Oct. 30, 2023

(30) Foreign Application Priority Data
May 4, 2021 (EP) 21171925.7

(51) Int. Cl. A61M 5/32

(52) U.S. Cl. CPC A61M 5/3204

(57) ABSTRACT

A needle shield remover (1) for removing a needle shield (11g) covering a needle of a medicament delivery device, the needle shield remover (1) comprising: a body (3) having a proximal end and a distal end, the body (3) comprising: an outer cap structure (5) having a distal end opening configured to receive a proximal end of a medicament delivery device, an inner cap structure (7) arranged radially inwards of and concentrically with the outer cap structure (5), the inner cap structure (7) having a central channel (7a); and a frustoconical grabber (9) having a top and a base, the grabber (9) being arranged in the central channel (7a) with the base facing the distal end of the body (3), the grabber (9) being configured to receive a proximal end of the needle shield, wherein the top is provided with a plurality of sharp edges configured to engage with and prevent the needle shield from rotating in a first direction relative to the grabber (9).

### TECHNICAL FIELD

The present disclosure generally relates to a needle shield remover for removing a needle shield covering a needle of a medicament delivery device.

### BACKGROUND

Prefilled syringes can be provided in a medicament delivery device such as an auto-injector or a manual injector. Syringes placed in a medicament delivery device often have a flexible needle shield (FNS) covering the needle, for keeping the needle sterile. The FNS may typically be made of a rubber material. The FNS may be covered by a rigid needle shield (RNS) to protect the FNS. The RNS and the FNS are removed from the syringe before the medicament contained in the syringe is to be delivered.

WO2020/039009 discloses a shield remover for a syringe. The shield remover has an outer cap and an intermediary component, or insert, coupled between the outer cap and the needle shield. The intermediary component has a shield engagement grip means for coupling the intermediary component with the needle shield so that when the shield remover is pulled away from the syringe the intermediary component exerts force against the needle shield to remove the needle shield from the syringe. The insert may be a tubular drum-shaped object retained in the outer cap due to the shape of the insert. The insert has segments which end in a barb, bent radially inwards engage and cut slightly into the rigid needle shield of the syringe when a user attempts to remove the outer cap from the housing.

### SUMMARY

An object of the present disclosure is to provide a needle shield remover which solves, or at least mitigates problems of the prior art.

There is hence provided a needle shield remover for removing a needle shield covering a needle of a medicament delivery device, the needle shield remover comprising: a body having a proximal end and a distal end, the body comprising: an outer cap structure having a distal end opening configured to receive a proximal end of a medicament delivery device, an inner cap structure arranged radially inwards of and concentrically with the outer cap structure, the inner cap structure having a central channel; and a frustoconical grabber having a top and a base, the grabber being arranged in the central channel with the base facing the distal end of the body, the grabber being configured to receive a proximal end of the needle shield, wherein the top is provided with a plurality of sharp edges configured to engage with and prevent the needle shield from rotating in a first direction relative to the grabber.

The needle shield remover may be especially beneficial for use with syringes which have a needle hub that needs to be twisted in the first direction to cause a double-edged needle to penetrate the syringe septum before use. The needle shield remover can however also be used with other types of syringes.

When the present needle shield remover is rotated in the first direction relative to a needle shield arranged in a medicament delivery device having a housing to which the needle shield remover is attached, it causes the needle shield to rotate concurrently with the needle shield remover, enabling penetration of the septum. Moreover, the grabber engages with and cuts into the needle shield when the needle shield remover is pulled from a medicament delivery device. Removal of the needle shield remover from the medicament delivery device thus also removes the needle shield.

With “sharp edges” is herein meant that the edges are configured to be able to engage with the needle shield by cutting into the external surface of the needle shield when the needle shield remover is rotated in the first direction relative to the medicament delivery device containing a syringe with the needle shield, and/or when the needle shield remover is pulled from the medicament delivery device.

The needle shield may comprise an FNS and an RNS covering the FNS.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to one embodiment the central channel has an inner surface provided with an axially extending rib and the grabber has a slit engaging with the rib to prevent rotation of the grabber relative to the inner cap structure.

According to one embodiment the inner cap structure is provided with a mounting stop extending radially inwards relative to an inner surface of the central channel, the mounting stop being configured to prevent the grabber from moving out axially from the central channel in a direction towards the distal end of the body.

According to one embodiment the mounting stop is arranged axially aligned with the rib.

According to one embodiment the inner cap structure has a distal ledge extending radially inwards in the inner channel, and wherein the grabber has a slot configured to engage with the ledge to restrict axial movement of the grabber towards the proximal end of the body.

According to one embodiment the sharp edges point towards the proximal end of the body and provide a ratchet function on the needle shield. The sharp edges thus engage with the needle shield when the needle shield remover is rotated in the first direction and allow relative rotation between the needle shield remover and the needle shield in a second direction opposite to the first direction.

According to one embodiment the grabber is mechanically flexible and configured to flex radially outwards by pressure provided by the needle shield.

According to one embodiment the grabber comprises metal.

According to one embodiment the central channel defines a proximal opening at the proximal end of the body.

According to one embodiment the inner cap structure has a shorter axial extension in a direction from the proximal end to the distal end than the outer cap structure.

According to one embodiment the central channel is cylindrical.

There is according to a second aspect provided a medicament delivery device comprising a needle shield remover according to the first aspect.

The medicament delivery device may comprise a syringe having a needle hub comprising a double-edged needle and a septum arranged distally relative to a distally pointing needle end.

The syringe may comprise a medicament container arranged distally relative to the septum. The needle hub may be configured to be rotated relative to medicament container to cause the needle hub to move in the distal direction and the distally pointing needle end to penetrate the septum.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 shows a perspective view of an example of a needle shield remover;

FIG. 2 shows another view of the needle shield remover in FIG. 1;

FIG. 3 shows a perspective view of an example of a grabber;

FIG. 4 shows a longitudinal section of the needle shield in FIG. 1;

FIG. 5 shows an example of a syringe;

FIG. 6 shows a perspective view of an example of a medicament delivery device provided with the needle shield remover in FIG. 1;

FIG. 7 is a longitudinal section of the medicament delivery device in FIG. 6;

FIG. 8A-9 show other examples, not within the scope of the claims, of needle shield removers.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

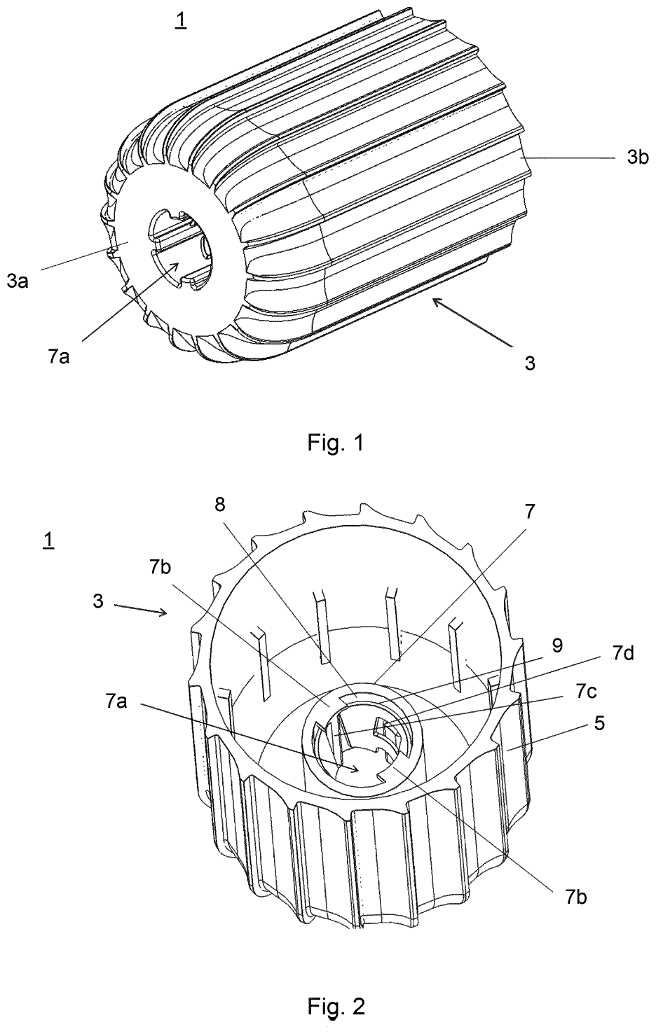

FIG. 1 shows a perspective view of an example of a needle shield remover 1. The needle shield remover 1 is configured to be fitted to a proximal end of a housing of a medicament delivery device provided with a syringe comprising a needle shield.

The needle shield remover 1 is configured to engage with the needle shield and remove the needle shield from the syringe when the needle shield remover 1 is removed from the housing. The needle shield remover 1 may be a cap.

The exemplified needle shield remover 1 is generally tubular. The needle shield remover 1 comprises a body 3 having a proximal end 3a and a distal end 3b. The needle shield remover 1 has a central axis extending between the proximal end 3a and the distal end 3b.

The exemplified needle shield remover has a central channel 7a which extends along the central axis. The central channel 7a defines a proximal opening at the proximal end 3a of the body 3.

FIG. 2 shows a perspective view of the needle shield remover 1. The body 3 comprises an outer cap structure 5 and an inner cap structure 7 arranged radially inside of and concentrically with the outer cap structure 5.

The outer cap structure 5 forms an outer wall of the body 3. The inner cap structure 7 forms an inner wall of the body 3. The inner cap structure 7 comprises the central channel 7a. The inner wall formed by the inner cap structure 7 has an inner surface 8 which defines the central channel 7a. The central channel 7a extends through the inner cap structure 7.

A radial space is formed between the outer wall and the inner wall. The outer wall and the inner wall are arranged to receive a proximal end portion of a housing of a medicament delivery device in the radial space.

The inner cap structure 7 has a shorter axial extension in a direction from the proximal end 3a to the distal end 3b than the outer cap structure 5.

The inner cap structure 7 comprises at least one mounting stop 7b extending radially inwards relative to the inner surface 8 of the central channel 7a. The at least one mounting stop 7b thus makes the central channel 7a narrower. The at least one mounting stop 7b may be arranged at a distal end of the central channel 7a.

The example shown in FIG. 2 comprises two mounting stops 7b extending towards each other. The mounting stops 7b are arranged oppositely relative to each other.

The inner surface 8 is provided with an axially extending rib 7c. The rib 7c may for example extend along a majority of the length of the central channel 7a. The mounting stop 7b may be arranged axially aligned with the rib 7c. The rib 7c may for example transition into the mounting stop 7b.

The inner surface 8 may have at least one distal ledge 7d extending radially inwards in the inner channel 7a. The distal ledge 7d comprises a radial surface directed towards the distal end 3b of the body 3. Thus, the inner surface 8 has a protrusion extending radially inwards, forming the distal ledge 7d.

The example described in FIGS. 1-4 comprises two distal ledges 7d arranged oppositely to each other.

The needle shield remover 1 comprises a grabber 9. The grabber 9 is an insert. The grabber 9 is arranged in the central channel 7a. The grabber 9 is configured to receive a proximal end of a needle shield.

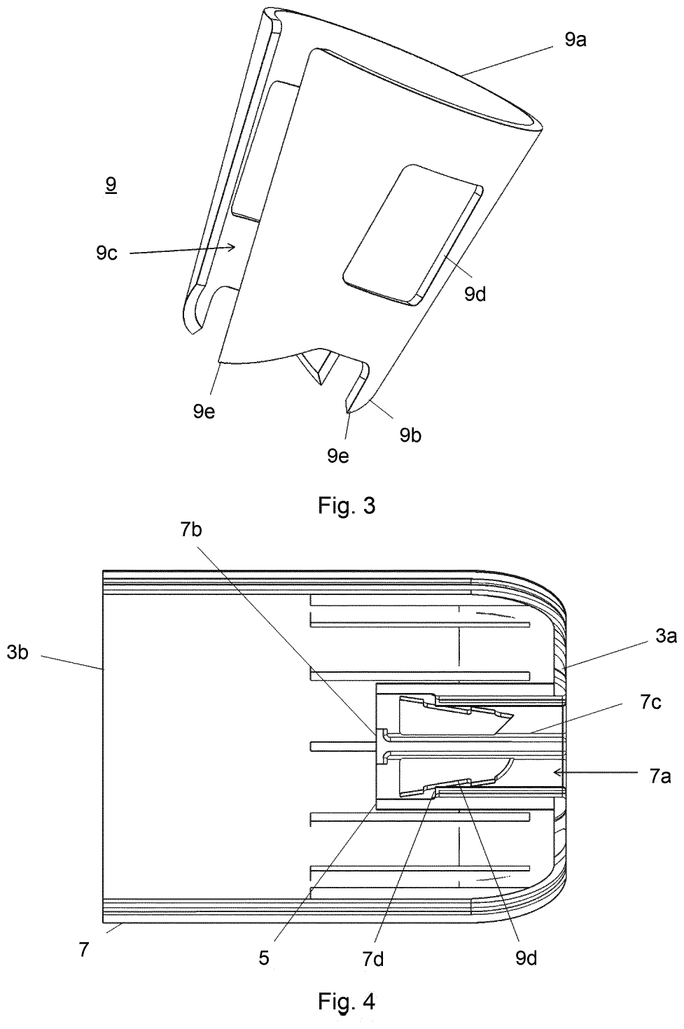

The grabber 9 is frustoconical, as shown in FIG. 3. The grabber 9 is inserted into the central channel 7a from the proximal end 3a of the body 3, through the proximal opening.

Turning now to FIG. 3, the frustoconical grabber 9 has a base 9a and a top 9b. The base 9a has a larger diameter than the top 9b. The base 9a faces the distal end 3b of the body 3. The top 9b faces the proximal end 3a of the body 3. The grabber 9 is inserted into the central channel 7a from the proximal end 3a of the body 3 with the base 9a first.

The grabber 9 comprises a slit 9c. The slit 9c extends along the longitudinal axis of the frustoconical grabber 9 all the way from the base 9a to the top 9b. The slit 9c divides the grabber 9 into two portions.

The slit 9c is configured to receive the rib 7c of the inner surface 8. The slit 9c and the rib 7c are configured to engage to prevent relative rotation between the grabber 9 and the inner cap structure 7.

The grabber 9 is mechanically flexible. The grabber 9 may have a larger outer diameter at the base 9a than the diameter of the inner surface 8. Due to the slit 9c, the two facing edges of the slit 9c may be pressed towards each other when the grabber 9 is inserted into the central channel 7a. The grabber 9 can thus be fitted inside the central channel 7a.

The mechanical flexibility provided by the slit 9c also enables the grabber 9 to adapt its size to the diameter of the needle shield when the diameter of the top 9b is made smaller than the outer diameter of the needle shield before the needle shield has been received by the grabber 9. The grabber 9 will thus be able to engage well with the needle shield.

The grabber 9 has at least one slot, or window, 9d configured to engage with the ledge 7d. In case of several slots 9d, each slot 9d is configured to engage with a respective one of the ledges 7d.

When the grabber 9 has been inserted into the central channel 7a, the distal edge of the slot 9d closest to the base 9a moves past the ledge 7d. The ledge 7d will therefore bear against the distal edge in case the grabber 9 would move towards the proximal end of the body 3. The ledge 7d restricts movement of the grabber 9 in the proximal direction and prevents the grabber 9 from moving out from the central channel 7a through the proximal opening.

The grabber 9 comprises a plurality of sharp edges 9e. The sharp edges 9e are provided at the top 9b. According to the example, the sharp edges 9e provide a ratchet functionality. The grabber 9 is configured to receive a proximal end of a needle shield and the sharp edges 9e are configured to engage with the needle shield when the grabber 9 is rotated in a first direction relative to the needle shield. The sharp edges 9e are configured to enable rotation of the grabber 9 relative to the needle shield in a second direction opposite the first direction.

The top 9b may have a sawtooth-like shape with the edges 9e being those of the sawtooth tips.

FIG. 4 shows a longitudinal section of the grabber 9. The grabber 9 is arranged in the central channel 7a. The ledges 7d prevent the grabber 9 to move further towards the proximal end 3a because they would engage with a respective one of the slots 9d.

The mounting stop 7b prevents the grabber 9 from moving out from the central channel 7a towards the distal end 3b. The top 9a would bear against the mounting stop 7b in case the grabber 9 is moved in the distal direction, and the grabber 9 would thus be prevented from further movement towards the distal end 3b.

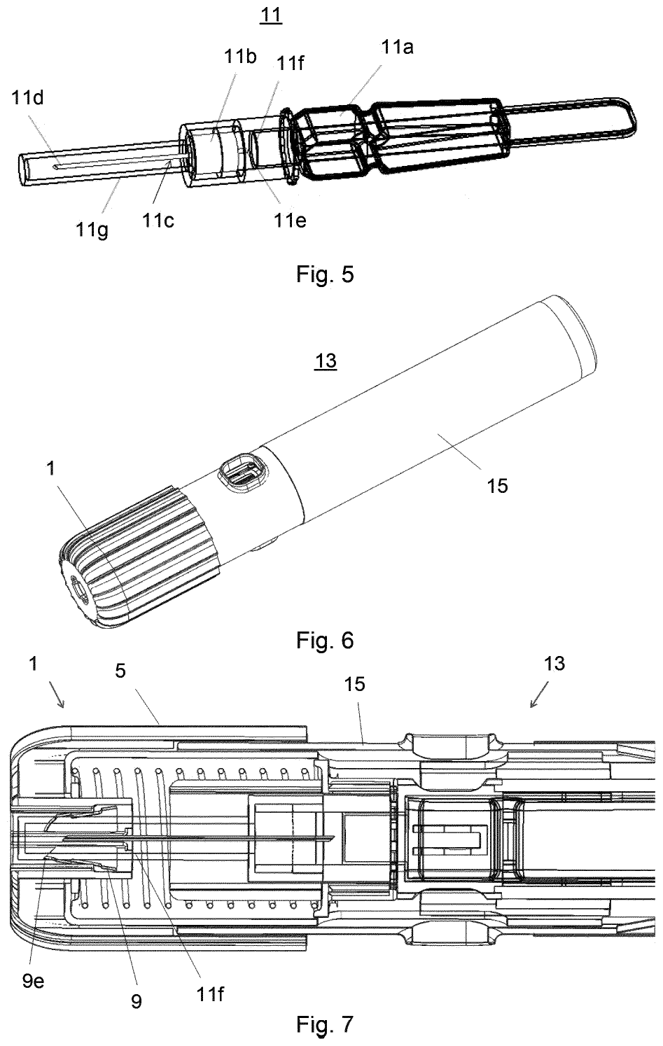

FIG. 5 shows a perspective view of a syringe 11. The syringe 11 in the example has a blow-fill-seal medicament container structure. The medicament container could however alternatively be of any other type.

The syringe 11 has a medicament container 11a containing a liquid medicament.

The syringe 11 comprises a needle hub 11b. The needle hub 11b is in the present example rotatably arranged relative to the syringe body.

The syringe 11 comprises a double-edged needle 11c fixedly arranged in the needle hub 11b. The double-edged needle 11c has a proximally pointing needle end 11d and a distally pointing needle end 11e. The needle could alternatively be single edged.

The syringe 11 comprises a septum 11f arranged between the distally pointing needle end 11d and the medicament container 11a. The septum 11f seals the medicament container and keeps the medicament sterile.

The syringe 11 comprises a needle shield 11g. The needle shield 11g may comprise an inner FNS directly covering the proximally pointing needle end 11d and an outer RNS arranged on the FNS. The RNS may be arranged tightly around the proximally pointing needle end 11d.

The needle shield remover 1 is configured to receive a proximal end portion of the needle shield 11g in the central channel 7a. In particular, the grabber 9 is configured to receive a proximal end portion of the needle shield 11g. Generally, the proximal end portion of the needle shield 11g extends through the grabber 9.

When the needle shield remover 1 is rotated in the first direction relative to the syringe 11, the sharp edges 9e engage with the needle shield 11g causing the needle shield 11g to rotate concurrently with the needle shield remover 1. The needle shield 11g is thus also rotated in the first direction. The syringe 11 is rotationally fixed in the medicament delivery device in which it has been installed. This causes the needle hub 11b to rotate relative to the medicament container 11a and move in the distal direction and the distally pointing needle end 11e to penetrate the septum 11f. The double-edged needle 11c thus comes into fluid communication with the medicament container 11a.

FIG. 6 shows an example of a medicament delivery device 13 comprising the needle shield remover 1. The medicament delivery device 13 may for example be an auto-injector or a manual injector.

The medicament delivery device 13 comprises a housing 15 configured to receive the syringe 11. The needle shield remover 1 is configured to be fitted to the proximal end of the housing 15, as shown in FIG. 6.

FIG. 7 shows a longitudinal section of a proximal end portion of the medicament delivery device 13 with the needle shield remover 1 attached to the housing 15. According to the example, a proximal end of the housing 15 extends into the outer cap structure 5, in the radial space between the outer wall and the inner wall.

The needle shield 11g extends into the central channel 7a and through the grabber 9. The needle shield 11g extends from the base 9a all the way to the top 9b and past the edges 9e towards the proximal end 3a of the body 3.

When the exemplified needle shield remover 1 is to be removed from the medicament deliver device 1, the user rotates the needle shield remover 1 in the first direction. As described above, this causes the needle shield 11g to rotate in the first direction too.

The needle shield remover 1 may for example comprises threads configured to threadedly engage with the housing 15. When the needle shield remover 1 is rotated in the first direction relative to the housing 15, the needle shield remover 1 may be unscrewed from the housing 15. Alternatively, the needle shield remover 1 could be provided without threads.

When the needle shield remover 1 is removed from the medicament delivery device 1 by twisting and/or pulling, the needle shield 11g is pulled from the double-edged needle 11c. The double-edged needle 11c is thus uncovered in the medicament delivery device 13, which is thus ready to be used.

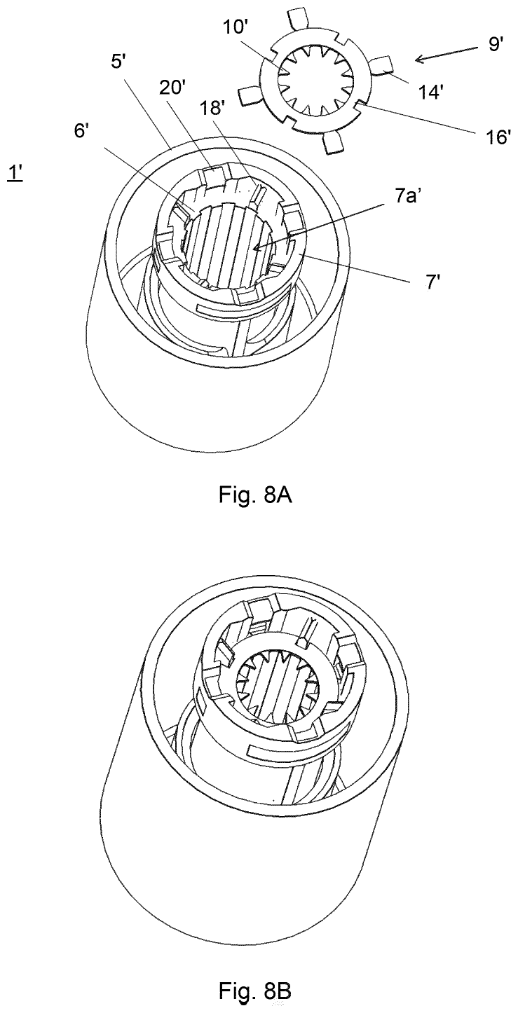

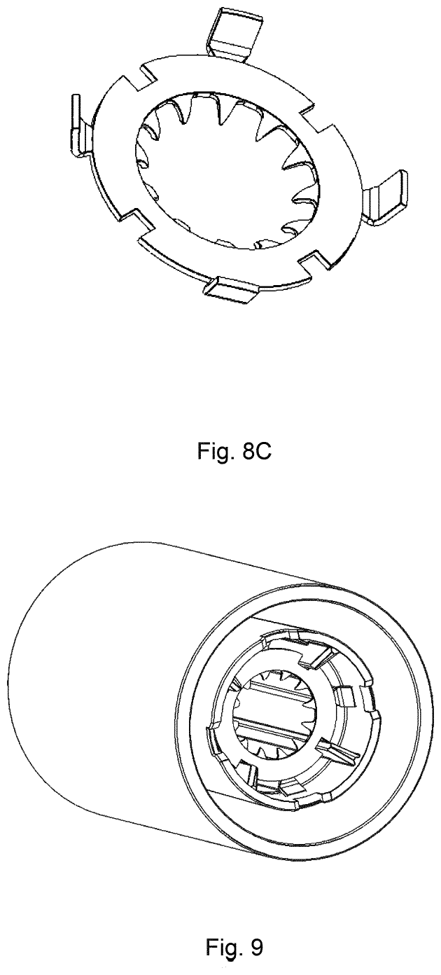

FIG. 8A shows another example of a needle shield remover. The needle shield remover 1′ comprises an outer cap structure 5′ and an inner cap structure 7′ arranged radially inwards of and concentrically with the outer cap structure 5′. The needle shield remover 1′ comprises a grabber 9′. The grabber 9′ is initially, i.e. before assembly, planar, as shown FIG. 8A.

The grabber 9′ has a through-opening defined by an inner surface provided with a plurality of radially inwards directed teeth 10′.

The grabber 9′ has a plurality of radially outwards extending guide structures 14′.

The exemplified grabber 9′ comprises at least one radially inwards extending recess 16′ provided in the outer perimeter of the grabber 9′.

The inner cap structure 7′ comprises a central channel 7a′. The inner cap structure 7′a is configured to receive the grabber 9′ in a seat structure 6′. The seat structure 6′ comprises at least one radially inwards extending rotation preventing structure 18′ configured to engage with a respective one of the at least one recess 16′. The grabber 9′ is thus prevented from rotation relative to the inner cap structure 7′.

The guide structures 14′ are used during mounting, when the grabber 9′ is inserted into the central channel 7a′ from the distal end of the needle shield remover 1′. The guide structure 14′ prevent the grabber 9′ from rotation during assembly. When the grabber 9′ rests with its guide structures 14′ in the corresponding guide recesses 20′ in the inner cap structure 7′, a needle shield may penetrate the through-opening of the grabber 9′. The guide structure 14′ is thereby deformed and the grabber 9′ will slide axially towards the proximal end of the needle shield remover 1′ until it reaches the seat structure 6′ where it will rest. The teeth 10′ will become deformed by this penetration, because the outer diameter of the needle shield is larger than the distance between oppositely arranged teeth 10′. The teeth 10′ will point towards the proximal end of the needle shield remover 1′. The teeth 10′ thus engages with the needle shield. The teeth 10′ prevent relative rotation between the needle shield remover and the grabber 9′. The teeth 10′ prevent axial movement in the proximal direction of the needle shield remover 9′ relative to the needle shield. Thus, when the grabber 9′ is removed from the medicament delivery device, it brings the needle shield with it.

FIG. 8B shows when the grabber 9′ has been installed in the inner cap structure 7′.

FIG. 8C shows the grabber 9′ after installation, when the teeth 10′ and the guide structures 14′ have been deformed.

In the example shown in FIGS. 8A-8B, the inner cap structure 7′ is removably attached to the outer cap structure 5′. The inner cap structure 7′ and the outer cap structure 5′ may for example be attached to each other by means of a threaded connection or by means of snap fits.

FIG. 9 shows a variation of the needle shield remover in FIGS. 8A-B, for which the inner cap structure and the outer cap structure are integral.

The inventive concept has mainly been described above with reference to a few examples. However, as is readily appreciated by a person skilled in the art, other embodiments than the ones disclosed above are equally possible within the scope of the inventive concept, as defined by the appended claims.

1. A needle shield remover (1) for removing a needle shield (11g) covering a needle (11c) of a medicament delivery device (13), the needle shield remover (1) comprising:
a body (3) having a proximal end (3a) and a distal end (3b), the body (3) comprising:
an outer cap structure (5) having a distal end opening configured to receive a proximal end of a medicament delivery device (13),
an inner cap structure (7) arranged radially inwards of and concentrically with the outer cap structure (5), the inner cap structure (7) having a central channel (7a); and
a frustoconical grabber (9) having a top (9b) and a base (9a), the grabber (9) being arranged in the central channel (7a) with the base (9a) facing the distal end (3b) of the body (3), the grabber (9) being configured to receive a proximal end of the needle shield (11g),
wherein the top (9b) is provided with a plurality of sharp edges (9e) configured to engage with and prevent the needle shield (11g) from rotating in a first direction relative to the grabber (9).

2. The needle shield remover (1) as claimed in claim 1, wherein the central channel (7a) has an inner surface (8) provided with an axially extending rib (7c) and the grabber (9) has a slit (9c) engaging with the rib (7c) to prevent rotation of the grabber (9) relative to the inner cap structure (7).

3. The needle shield remover (1) as claimed in claim 2, wherein the inner cap structure (7) is provided with a mounting stop (7b) extending radially inwards relative to an inner surface (8) of the central channel (7a), the mounting stop (7b) being configured to prevent the grabber (9) from moving out axially from the central channel (7a) in a direction towards the distal end of the body (3).

4. The needle shield remover (1) as claimed in claim 3, wherein the mounting stop (7b) is arranged axially aligned with the rib (7c).

5. The needle shield remover (1) as claimed in claim 1, wherein the inner cap structure (7) has a distal ledge (7d) extending radially inwards in the inner channel (7a), and wherein the grabber (9) has a slot (9d) configured to engage with the ledge (7d) to restrict axial movement of the grabber (9) towards the proximal end (3a) of the body (3).

6. The needle shield remover (1) as claimed in claim 1, wherein the sharp edges (9e) point towards the proximal end (3a) of the body (3) and provide a ratchet function on the needle shield (11g).

7. The needle shield remover (1) as claimed in claim 1, wherein the grabber (9) is mechanically flexible and configured to flex radially outwards by pressure provided by the needle shield (11g).

8. The needle shield remover (1) as claimed in claim 1, wherein the grabber (9) comprises metal.

9. The needle shield remover (1) as claimed in claim 1, wherein the central channel (7a) defines a proximal opening at the proximal end (3a) of the body (3).

10. The needle shield remover (1) as claimed in claim 1, wherein the inner cap structure (7) has a shorter axial extension in a direction from the proximal end (3a) to the distal end (3b) than the outer cap structure (5).

11. The needle shield remover (1) as claimed in claim 1, wherein the central channel (7a) is cylindrical.

12. The needle shield remover (1) as claimed in claim 1, wherein the frustoconical grabber (9) is configured to engage with and cut into the needle shield (11g) when the needle shield remover (1) is pulled from a medicament delivery device (13), so that removal of the needle shield remover (1) from the medicament delivery device (13) thus also removes the needle shield (11g).

13. The needle shield remover (1) as claimed in claim 1, wherein the plurality of sharp edges (9e) are configured to engage with and prevent the needle shield (11g) from rotating in a first direction relative to the grabber (9) by cutting into an external surface of the needle shield (11g) when the needle shield remover (1) is rotated in the first direction relative to a medicament delivery device (13) containing a syringe with the needle shield (11g), and/or when the needle shield remover (1) is pulled from the medicament delivery device (13).

14. A medicament delivery device (13) comprising a needle shield remover (1) as claimed in claim 1.
